(19) United States
(12) Patent Application Publication
(10) Pub. No.: US 2014/0069426 A1
(43) Pub. Date: Mar. 13, 2014

(54) SYSTEMS AND METHODS FOR ANESTHETIZING A RODENT

(71) Applicants: James Thornton Houts, Bend, OR (US); Ryan Gregory Sullivan, Half Moon Bay, CA (US)

(72) Inventors: James Thornton Houts, Bend, OR (US); Ryan Gregory Sullivan, Half Moon Bay, CA (US)

(21) Appl. No.: 13/960,074

(22) Filed: Aug. 6, 2013

Related U.S. Application Data
(60) Provisional application No. 61/700,656, filed on Sep. 13, 2012.

(51) Int. Cl. A61D 7/04; A61D 3/00; A61M 16/06; A61B 19/00; A61M 16/00; A61M 16/10

(52) U.S. Cl. CPC A61D 7/04; A61M 16/009; A61M 16/0057; A61M 16/104; A61M 16/06; A61B 19/203; A61D 3/00
USPC 128/203.29

(57) ABSTRACT

The present disclosure includes a method comprising positioning a nose of a rodent within a nose chamber of a device for anesthetizing the rodent, the nose chamber configured to partially enclose an anesthetizing gas and hold the nose of the rodent in contact with the anesthetizing gas, delivering a positive flow of the anesthetizing gas into the nose chamber through a gas intake channel sufficient to anesthetize the rodent, supplying a waste gas reservoir to collect the anesthetizing gas, the waste gas reservoir in communication with the nose chamber through a gas transport channel and coupled with the nose chamber in an open configuration allowing the anesthetizing gas and ambient air to flow into the waste gas reservoir, and actively retrieving the anesthetizing gas and ambient air from the waste gas reservoir with a negative flow conveyed through a gas outtake channel in communication with the waste gas reservoir.

### PRIORITY INFORMATION

The present application claims priority under 35 U.S.C. §119(e) to U.S. Provisional Patent Application Ser. No. 61/700,656, filed Sep. 13, 2012 and incorporated by reference herein.

### TECHNICAL FIELD OF THE INVENTION

The present invention relates generally to research tools and, more particularly, to systems and methods for anesthetizing a rodent.

### BACKGROUND

Research in a variety of areas has been facilitated by using animal models for testing and research. This may be particularly beneficial because of the variability introduced through biological systems. Animal testing may be used in diverse fields of endeavor. For example, animal testing may be used in the biomedical industry to model or test surgical techniques, medical devices, or drug treatments. Animal testing may be used in behavioral studies, for example, to observe mice running mazes or the social interactions of chimpanzees. Animal testing may be used for genetic studies, for example, to determine dominant and recessive genes and the passing of that gene between generations. Animal testing may also be used for instructive purposes, for example, to allow a student to observe first-hand the skeletal and muscular structure of a frog, or to observe the force generated by the contraction of a muscle.

One type of animal testing that may be beneficial is stereotactic (or stereotaxic) surgery. In such a surgery, a three-dimensional coordinate system may be used to locate small targets and perform some task on that target. One frequent use of stereotactic surgery is to perform some task on a portion of the brain. Some stereotactic surgical devices have been sized and constructed to perform stereotactic surgeries on rodents. In such a surgery, the subject is typically anesthetized.

### SUMMARY

In one embodiment, the present disclosure describes a device for anesthetizing a rodent comprising a nose chamber configured to at least partially enclose an anesthetizing gas and to position and hold in contact with the anesthetizing gas a nose of the rodent, and a gas intake channel in communication with the nose chamber and configured to deliver a positive flow of the anesthetizing gas into the nose chamber, the positive flow sufficient to anesthetize the rodent. The device also comprises a waste gas reservoir configured to collect the anesthetizing gas, the waste gas reservoir coupled with the nose chamber in an open configuration allowing the anesthetizing gas and ambient air to flow into the waste gas reservoir, and a gas transport channel configured to conduct the anesthetizing gas from the nose chamber to the waste gas reservoir. The device additionally comprises a gas outtake channel in communication with the waste gas reservoir and configured to convey a negative flow that actively retrieves the anesthetizing gas and ambient air from the waste gas reservoir.

In another embodiment, the present disclosure includes a method comprising positioning a nose of a rodent within a nose chamber of a device for anesthetizing the rodent, the nose chamber configured to at least partially enclose an anesthetizing gas and to hold the nose of the rodent in contact with the anesthetizing gas and delivering a positive flow of the anesthetizing gas into the nose chamber through a gas intake channel sufficient to anesthetize the rodent. The method additionally comprises supplying a waste gas reservoir to collect the anesthetizing gas, the waste gas reservoir in communication with the nose chamber through a gas transport channel and coupled with the nose chamber in an open configuration allowing the anesthetizing gas and ambient air to flow into the waste gas reservoir, and actively retrieving the anesthetizing gas and ambient air from the waste gas reservoir with a negative flow conveyed through a gas outtake channel in communication with the waste gas reservoir.

In an additional embodiment, the present disclosure includes a system comprising a stereotactic surgical device and a device for anesthetizing a rodent coupled to the stereotactic surgical device. The device for anesthetizing a rodent comprises a nose chamber configured to at least partially enclose an anesthetizing gas and to position and hold in contact with the anesthetizing gas a nose of the rodent, and a gas intake channel in communication with the nose chamber and configured to deliver a positive flow of the anesthetizing gas into the nose chamber, the positive flow sufficient to anesthetize the rodent. The device for anesthetizing a rodent further comprises a waste gas reservoir configured to collect the anesthetizing gas, the waste gas reservoir coupled with the nose chamber in an open configuration allowing the anesthetizing gas and ambient air to flow into the waste gas reservoir, and a gas transport channel configured to conduct the anesthetizing gas from the nose chamber to the waste gas reservoir. The device for anesthetizing a rodent additionally comprises a gas outtake channel in communication with the waste gas reservoir and configured to convey a negative flow that actively retrieves the anesthetizing gas and ambient air from the waste gas reservoir.

### BRIEF DESCRIPTION OF THE DRAWINGS

For a more complete understanding of the present disclosure and its features and advantages, reference is now made to the following description, taken in conjunction with the accompanying drawings, in which:

FIGS. 1A-1D illustrate an example embodiment of a device for anesthetizing a rodent, in accordance with the present disclosure;

FIGS. 2A-2C illustrate an example embodiment of a mask component of a device for anesthetizing a rodent, in accordance with the present disclosure;

FIGS. 3A and 3B illustrate an example embodiment of a gas gathering component of a device for anesthetizing a rodent, in accordance with the present disclosure; and

FIG. 4 illustrates an example embodiment of a stereotactic surgical system in accordance with the present disclosure.

### DETAILED DESCRIPTION

The present disclosure relates to systems and methods for anesthetizing a rodent. For example, the present disclosure may include a device configured to provide an anesthetizing gas in a sufficient amount to anesthetize the rodent, while at the same time removing any waste gases to a level sufficient to address exposure concerns for human operators of the device. For example, the present disclosure may facilitate a researcher performing a stereotactic surgery on a rodent by anesthetizing the rodent but preventing the researcher from being excessively exposed to the anesthetizing agent for the rodent.

As used herein, the term rodent may typically refer to mice and/or rats of any breed, strain, or variation, whether naturally occurring or genetically modified for research purposes. In addition to mice and/or rats, other rodents may be included that are of a comparable size and weight, for example, guinea pigs, hamsters, and chinchillas. As used herein, the term communication may refer to fluid communication, meaning that a fluid (like a gas) may flow between the two elements in communication.

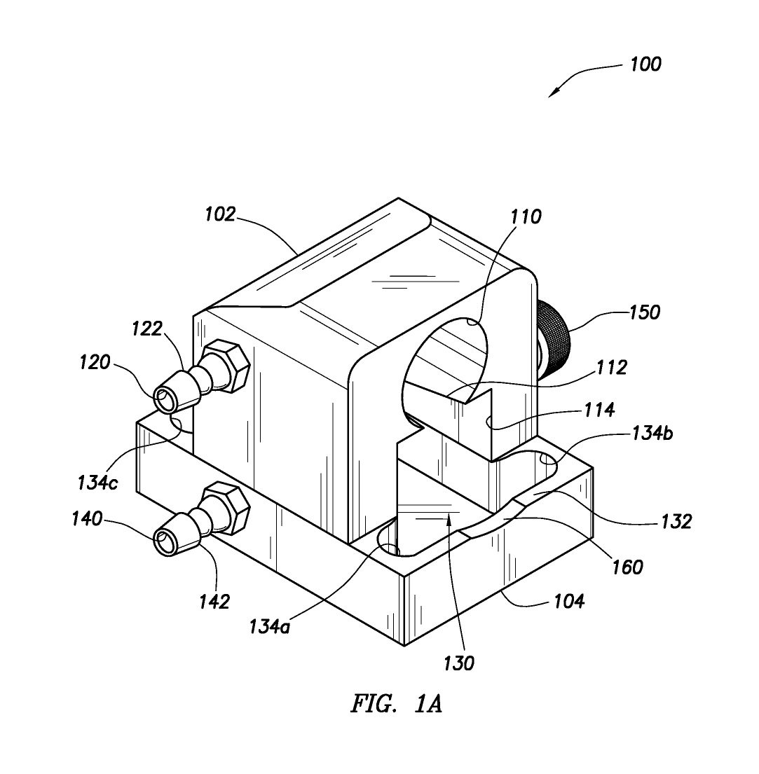

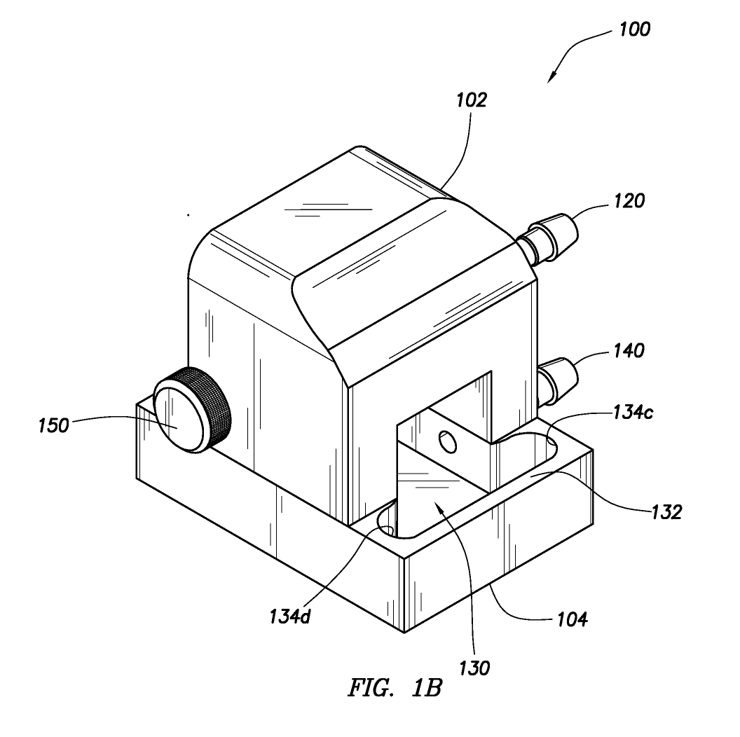

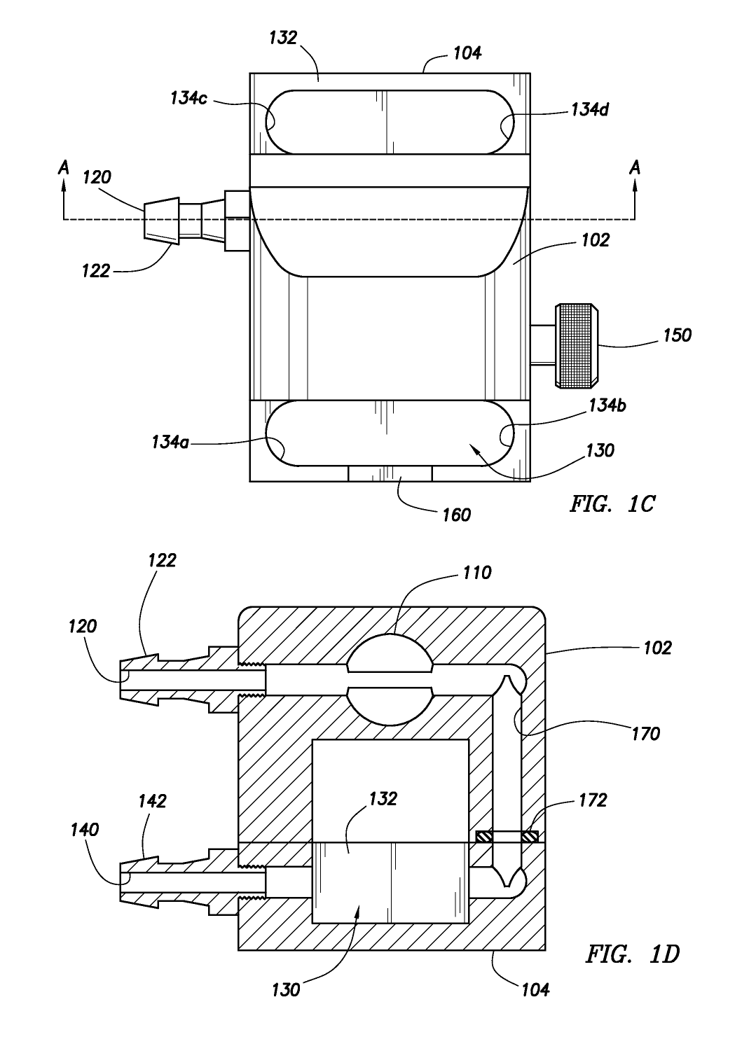

FIGS. 1A-1D illustrate an example embodiment of a device 100 for anesthetizing a rodent. FIG. 1A provides a perspective view of device 100. FIG. 1B provides an alternative perspective view of device 100. FIG. 1C provides a top view of device 100. FIG. 1D provides a sectioned view of device 100 along plane A (as shown in FIG. 1C).

Device 100 for anesthetizing a rodent may comprise a mask component 102 and a gas gathering component 104. Mask component 102 may be configured to deliver anesthetizing gas to the nose of a rodent, and gas gathering component 104 may be configured to retrieve waste gases. Gas gathering component 104 may be positioned directly beneath mask component 102, and may be larger than mask component 102 to facilitate the retrieval of waste gas. These two components may be a unitary body, or may be individually manufactured and later attached in a permanent or semi-permanent arrangement. For example, they may be attached using an adhesive such as glue, epoxy, or cement, or they may be attached using mechanical fasteners like screws, rivets, nuts and bolts, or the like. Device 100 may be machined or cast from Delrin®, aluminum, stainless steel, titanium, hard plastic, or other suitable material. Mask component 102 and gas gathering component 104 need not be made of the same material, but they may be.

Mask component 102 of device 100 may include a nose chamber 110 that is shaped and configured to receive a nose of a rodent. For example, nose chamber 110 may be a frustoconical shape to accommodate the tapering snout of a rodent. Alternatively, nose chamber 110 may be cylindrical, ellipsoidal, or any other shape that may receive the nose of the rodent. In some embodiments, the shape of nose chamber 110 may be selected so as to more appropriately direct and contain an anesthetizing gas in proximity to a nose of a rodent that has been positioned within nose chamber 110 such that the rodent will receive a sufficient amount of anesthetizing gas to anesthetize the rodent. A frustoconical shape may be useful to perform this function, but other shapes maybe used. Nose chamber 110 may at least partially enclose the anesthetizing gas while holding the nose of the rodent in contact with the anesthetizing gas. As can be seen from FIGS. 1B and 1D, nose chamber 110 may not pass entirely through mask component 102, but may only extend partially through mask component 102.

Mask component 102 of device 100 may also include an incisor channel 114. Incisor channel 114 may be configured to receive any portions of a rodent's head that may extend below nose chamber 110 when the rodent's nose is positioned in nose chamber 110, for example, incisors or other teeth, lower jaw, chin, or other facial features of a rodent. Incisor channel 114 may also be configured to receive an incisor bar of a stereotactic surgical device. To this end, as can be seen in FIGS. 1A, 1B, and 1D, incisor channel 114 may pass completely through device 100 to allow the incisor bar to remain attached to the stereotactic surgical device while at the same time allowing device 100 to slide down the incisor bar when coupling to the stereotactic surgical device. Incisor channel 114 may be in communication with nose chamber 110 via an opening 112.

Opening 112 may be shaped and configured to allow gases to flow from nose chamber 110 into incisor channel 114. For example, opening 112 may be wedge-shaped, rounded wedge-shaped, parabolic, rectangular, or any other shaped opening that maintains sufficient anesthetizing gas in nose chamber 110 to anesthetize a rodent while allowing device 100 to be coupled to a stereotactic surgical device. In some embodiments, an incisor bar may extend from incisor channel 114 through opening 112 and into nose chamber 110. In such an embodiment, portions of the rodent may or may not extend down into incisor channel 114. In some embodiments, at least some portion of anesthetizing gas that is delivered to nose cone 110 may pass through opening 112 and into incisor channel 114.

Device 100 may also include a gas intake channel 120. Gas intake channel 120 may be configured to deliver anesthetizing gas from an anesthetizing gas source to a rodent whose nose is disposed within nose cone 110. For example, as shown in FIG. 1D, gas intake channel 120 may pass partially through mask component 102 of device 100 until it reaches nose chamber 110, delivering the anesthetizing gas to nose chamber 110. In some embodiments, the anesthetizing gas may be delivered via gas intake channel 120 in a controlled positive flow rate. This flow rate may be selected to be sufficient to anesthetize a rodent based on the anesthetizing gas used, the size of gas intake channel 120, and the weight of the rodent. In some embodiments, the positive flow rate may be between about four hundred and fifty cubic centimeters per minute (450 cc/min) and about one thousand one hundred cc/min (1,100 cc/min). In some embodiments, the positive flow rate may be between about five hundred cc/min (500 cc/min) and about one thousand cc/min (1,000 cc/min). In some embodiments, the positive flow rate may be about five hundred cc/min (500 cc/min). In some embodiments, gas intake channel 120 may be around 0.2 inches in diameter. However, gas intake channel 120 may be any size so long as it does not excessively obstruct the airflow rate and is able to deliver sufficient gas to anesthetize the rodent.

Gas intake channel 120 may be coupled to a hose barb 122 for attaching a hose from the anesthetizing gas source to the gas intake channel 120. For example, hose barb 122 may be configured to screw into threads of gas intake channel 120 on one end and connect to the hose via a friction fit on the other end. Protrusions of hose barb 122 may resist withdrawal of a hose attached to hose barb 122 to maintain a secure connection between the hose and hose barb 122. In some embodiments, the hose may be coupled to hose barb 122 via a threaded connection, a clamp, or some other attachment mechanism. Similarly, hose barb 122 may be coupled to gas intake channel 120 via some other connection besides a threaded connection, for example, a friction fit, a compression fit, an adhesive coupling, or some other attachment mechanism. The hose may be 0.25-inch inner-diameter hose, but any size hose may be used that does not excessively obstruct the airflow rate and delivers sufficient gas to anesthetize the rodent. For example, 0.125-inch inner-diameter hose may be used. Gas gathering component 104 of device 100 may comprise a waste gas reservoir 130 for collecting and gathering of waste gas. Waste gas reservoir 130 may be positioned directly beneath nose chamber 110, and may be in direct and open communication with incisor channel 114 and open communication with nose chamber 110. In other words, gases may flow freely from nose chamber 110 through incisor channel 114 and into waste gas reservoir 130. Most anesthetizing gases, for example, isoflurane, are heavier than ambient air so the anesthetizing gas may settle into waste gas reservoir 130. For example, as described previously, some anesthetizing gas may flow from nose chamber 110 through opening 112 and into incisor channel 114 because of the open configuration of device 100. The anesthetizing gas in channel 114 may then settle into waste gas reservoir 130. Waste gas reservoir 130 may comprise an open collection area encircled by a raised boundary 132 to prevent the escape of anesthetizing gas from waste gas reservoir 130.

As shown in FIGS. 1A, 1B, and 1C, waste gas reservoir 130 may extend beyond both ends of incisor channel 114, which may pass completely through mask component 102. In some embodiments, waste gas reservoir may extend beyond incisor channel 114 on one of the ends, or neither end. Waste gas reservoir 130 may further include recesses 134a-d. Recesses 134a-d may be configured to allow further capture of any escaping anesthetizing gas. For example, anesthetizing gas escaping peripherally from incisor channel 114 or nose chamber 110 may settle into recesses 134a-d of waste gas reservoir 130. Further, recesses 134a-d may allow ambient air greater access to waste gas reservoir 130.

Gas gathering component 104 of device 100 may further comprise a gas outtake channel 140. Gas outtake channel 140 may be configured to remove anesthetizing gas from waste gas reservoir 130. For example, as shown in FIG. 1D, gas outtake channel 140 may pass partially through gas gathering component 104 of device 100, extending through raised boundary 132 and into waste gas reservoir 130. In some embodiments, waste gas may be actively retrieved via gas outtake channel 140 in a controlled negative flow rate. This may be provided, for example, by a vacuum system, a non-recirculating vent, or some other negative flow-rate mechanism. This flow rate may be selected to be sufficient to remove anesthetizing gas from the waste gas reservoir and surrounding ambient air in an amount sufficient to ensure that a human operator of device 100 will be exposed less than a maximum amount of the anesthetizing gas that is safe for human consumption. For example, in some embodiments, a human operator may only be exposed to the amount of anesthetizing gas allowed according to Occupational Safety and Health Administration (OSHA) standards. For example, in some embodiments, a human operator may be exposed to less than two parts per million per hour (2 ppm/hr) of anesthetizing gas. The negative flow rate may also be selected such that it does not actively retrieve the anesthetizing gas too quickly or too aggressively thereby preventing the rodent from receiving sufficient anesthetizing gas to be anesthetized. For example, if the negative flow rate were too high, the rodent may not be exposed to a sufficient amount of anesthetizing gas and may not be anesthetized. This in turn may prevent the performance of the stereotactic surgery.

In some embodiments, the negative flow rate may be between about nine L/min (9 L/min) and about twenty L/min (20 L/min). In some embodiments, the negative flow rate may be between about ten L/min (10 L/min) and about fifteen L/min (15 L/min). In some embodiments, the negative flow rate may be about fifteen L/min (15 L/min). In some embodiments, gas outtake channel 140 may be around 0.2 inches in diameter. However, gas outtake channel 140 may be any size so long as it does not excessively obstruct the airflow rate and allows removal of sufficient gas to ensure that a human operator of device 100 will only be exposed to an acceptable amount of anesthetizing gas.

Gas outtake channel 140 may be coupled to a hose barb 142 for attaching a hose from the negative flow source to the gas outtake channel 140. For example, hose barb 142 may be configured to screw into threads of gas outtake channel 140 on one end and connect to hose via a friction fit on the other end. Protrusions of hose barb 142 may resist withdrawal of a hose attached to hose barb 142 to maintain a secure connection between the hose and hose barb 142. In some embodiments, the hose may be coupled to hose barb 142 via a threaded connection, a clamp, or some other attachment mechanism. Similarly, hose barb 142 may be coupled to gas outtake channel 140 via some other connection besides a threaded connection, for example, a friction fit, a compression fit, an adhesive coupling, or some other attachment mechanism. The hose may be 0.25-inch inner-diameter hose, but any size hose may be used that does not excessively obstruct the airflow rate and allows removal of sufficient gas to ensure that a human operator of device 100 will only be exposed to an acceptable amount of anesthetizing gas. For example, 0.125-inch inner-diameter hose may be used.

Device 100 additionally may comprise a thumbscrew 150 to facilitate attachment of device 100 to a stereotactic surgical device. For example, thumbscrew 150 may pass through mask component 102 and engage with an incisor bar or some other feature of a stereotactic surgical device to stabilize and maintain the position of device 100 relative to the stereotactic surgical device. It will be appreciated that any other means of attaching and/or securing device 100 to a stereotactic surgical device may be employed without detracting from the scope of the present invention.

Device 100 may further comprise an indentation 160 to facilitate a rodent interfacing with device 100. For example, a portion of a rodent's anatomy, for example a neck, shoulder, or chest may rest within indentation 160 when the rodent's nose is disposed within nose chamber 110.

As shown in FIG. 1D, device 100 may further comprise a gas transport channel 170. Gas transport channel 170 may be configured to convey anesthetizing gas from nose chamber 110 through gas transport channel 170 to waste gas reservoir 130. This may provide a number of benefits. For example, and in no way limiting, gas transport channel 170 may provide an exit path from nose chamber 110. Without such an exit path for anesthetizing gas, a seal may be created by the rodent's nose causing the anesthetizing gas to over-inflate the rodent's lungs, which may cause permanent damage to the rodent. As another non-limiting example, gas transport channel 170 may facilitate the collection of anesthetizing gas in waste gas reservoir 130 rather than allowing it to spill out into the ambient air. In some embodiments, gas transport channel 170 may be comparably sized to one of gas intake channel 120 and gas outtake channel 140, but need not be. In some embodiments, gas transport channel may span both mask component 102 and gas gathering component 104. If the two components are separately manufactured and later attached, a seal 172 may be placed around gas transport channel 170 to prevent the escape of anesthetizing gas travelling through gas transport channel 170 at the seam between mask component 102 and gas gathering component 104. Seal 172 may be made of any suitable material and may take any suitable form, for example, a rubber o-ring.

Using device 100, waste gas may be actively retrieved from waste gas reservoir 130. It will be appreciated that waste gas may come from a variety of sources. For example, waste gas may include directed anesthetizing gas that is conducted from nose chamber 110 to waste gas reservoir 130 through gas transport channel 170. Additionally, waste gas may include escaped anesthetizing gas that escapes from the nose chamber 110, through opening 112 into incisor channel 114, and then into waste gas reservoir 130. As another example, waste gas may include used anesthetizing gas that is exhaled from the rodent. As a further example, waste gas may include anesthetizing gas that has diffused into ambient air around device 100. In each of these cases, the waste gas may eventually arrive in waste gas reservoir 130 to be actively retrieved via gas outtake channel 140. Waste gas may also include any amount of ambient air, for example, that portion that may be actively collected by device 100 to ensure that a human user of the device will be exposed to less than a maximum amount of the anesthetizing gas safe for human consumption.

As described above, in some embodiments gas outtake channel 140 actively retrieves waste anesthetizing gas as well as ambient air when retrieving waste gas. For example, using the ranges described previously, gas intake channel 120 may providing gas at a rate of between four hundred and fifty cc/min (450 cc/min) and one thousand one hundred cc/min (1,100 cc/min), and gas outtake channel 140 may retrieving gas at a rate of between five L/min (5 L/min) and twenty L/min (20 L/min). Even at the highest provide rate, lowest retrieval rate, and complete retrieval of the anesthetizing gas, at least 3.9 L/min of ambient air is also being actively retrieved (5 L/min−1.1 L/min=3.9 L/min). By providing a larger degree of negative flow rate compared to the positive flow rate, ambient air may be collected, for example, through incisor channel 114 or recesses 134a-d, into waste gas reservoir 130 and out through gas outtake channel 140. In this way, even if a small amount of anesthetizing gas does diffuse into ambient air or escape outside of waste gas reservoir 130, there is an increased probability that the anesthetizing gas will still be actively retrieved. However, it will be appreciated that this negative flow must be balanced so as not to retrieve the anesthetizing gas too aggressively so the rodent is not anesthetized. Thus, in some embodiments the positive flow rate and negative flow rates are selected together to ensure that the delivery rate and retrieval rate are complimentary to anesthetize the rodent but ensure that a human user of device 100 will be exposed to less than a maximum amount of the anesthetizing gas safe for human consumption.

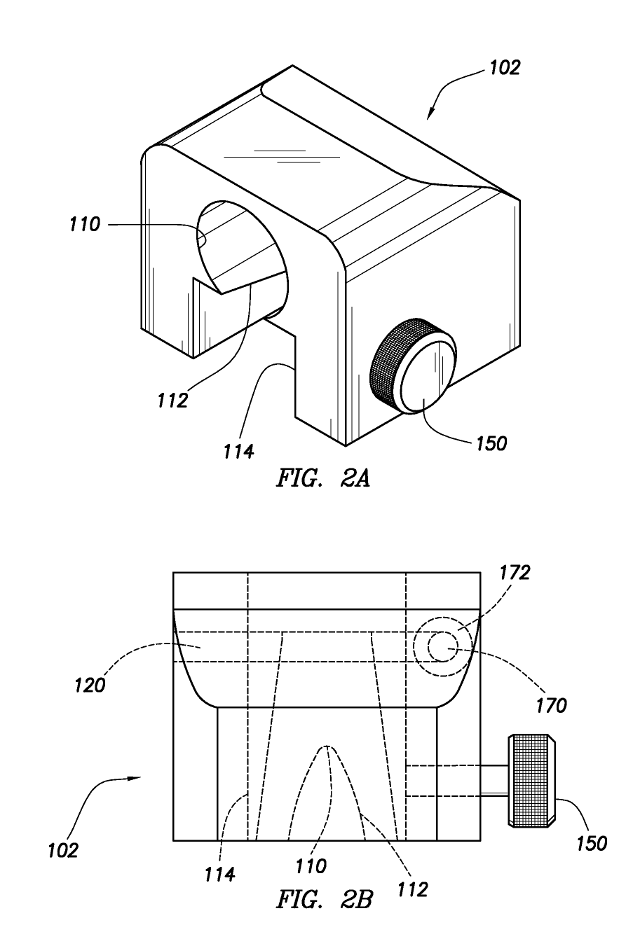

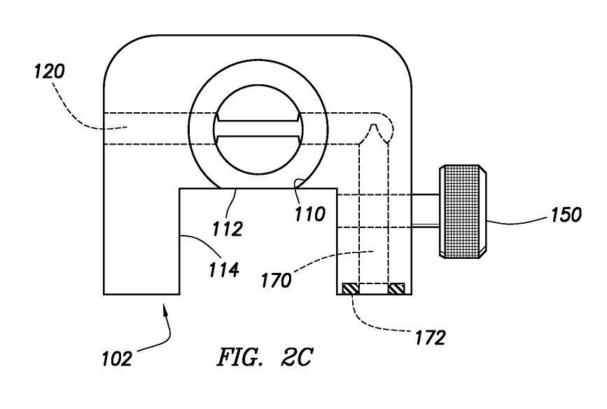

FIGS. 2A-2C illustrate an example embodiment of mask component 102 of device 100 for anesthetizing a rodent. FIG. 2A illustrates a perspective view of mask component 102. FIG. 2B illustrates a top view of mask component 102, with internal regions designated by dashed lines. For example, as shown in FIG. 2B, gas intake channel 120 extends to nose chamber 110. Nose chamber 110 does not extend completely through mask component 102. However, incisor channel 114 may pass completely through mask component 102. Additionally, an embodiment in which opening 112 is parabolic in shape is shown. Gas transport channel 170 is also shown extending from nose chamber 110 towards gas gathering component 104. FIG. 2C illustrates a view of mask component 102 looking into nose chamber 110. As can be seen, in this embodiment, nose chamber 110 is frustoconical in shape and meets gas intake channel 120.

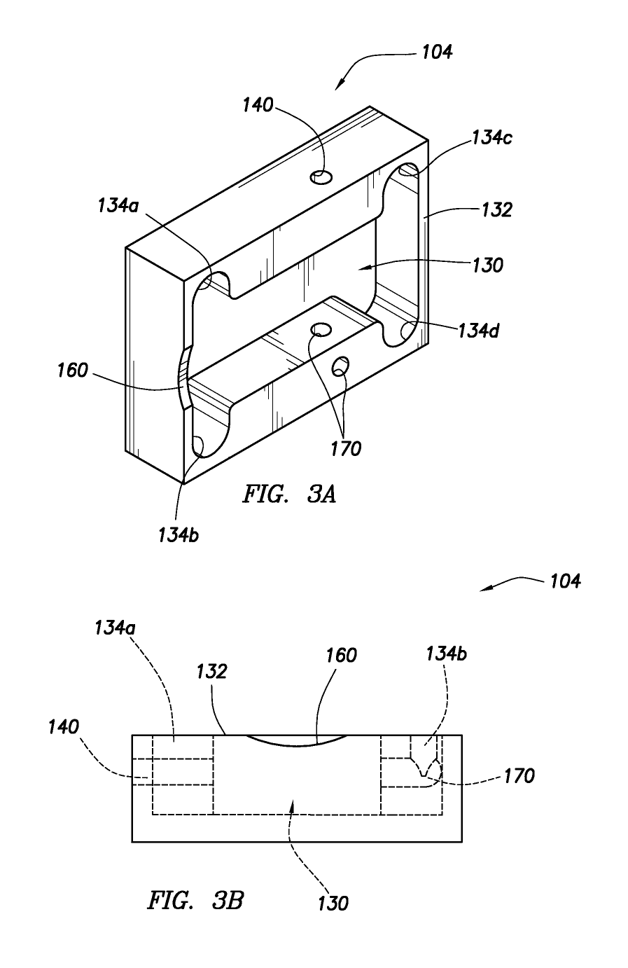

FIGS. 3A and 3B illustrate an example embodiment of gas gathering component 104 of device 100 for anesthetizing a rodent. FIG. 3A provides a perspective view of gas gathering component 104. FIG. 3B provides a view from the same perspective of that in FIG. 2C, except gas gathering component 104 is shown rather than mask component 102. As can be seen from FIG. 3B, gas transport channel 170 may convey anesthetizing gas from nose chamber 110 into waste gas reservoir 130. Gas outtake channel 140 may actively retrieve waste gas from waste gas reservoir 130 as gas outtake channel 140 passes completely through one side of raised boundary 132.

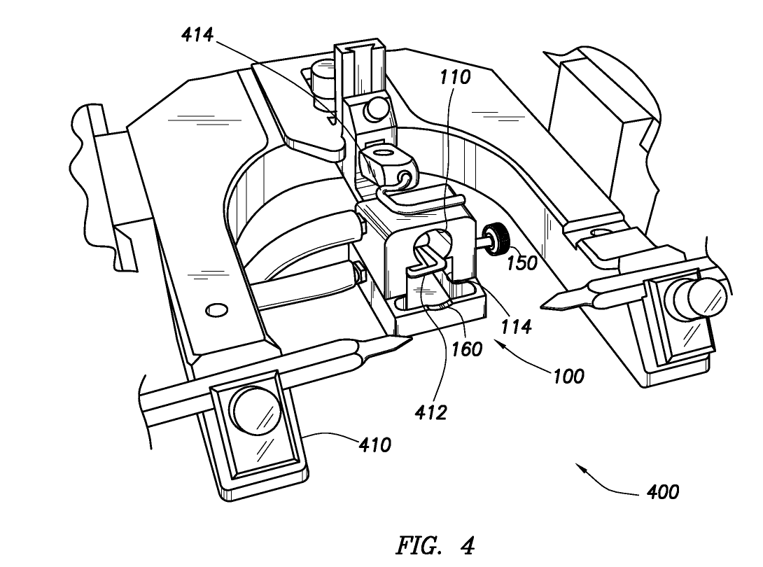

FIG. 4 illustrates an example embodiment of a stereotactic surgical system 400 in accordance with the present disclosure. As shown in FIG. 4, system 400 includes a stereotactic surgical device 410. Stereotactic surgical device 410 may be configured to facilitate performance of stereotactic surgeries. Stereotactic surgical device 410 may comprise an incisor bar 412 and a securing arm 414. As described above, device 100 for anesthetizing a rodent may be coupled with stereotactic surgical device 410. For example, device 100 may be provided. Incisor bar 412 may be passed or slid through incisor channel 114. In some embodiments, an end of incisor bar 412 may extend upwards into nose chamber 110. A rodent may then have its incisors placed over the end of the incisor bar. Device 100 may then be slid along incisor bar 412 until the rodent's nose may be positioned within nose chamber 110. The chest or neck of the rodent may rest in indentation 160. Thumbscrew 150 may then be used to fix device 100 to incisor bar 412, thus securing device 100 relative to stereotactic surgical device 410. In some embodiments, securing arm 414 may also be used to secure device 100 relative to stereotactic surgical device 410.

A positive flow of anesthetizing gas may then be delivered to the rodent's nose in nose chamber 110 via gas intake channel 120 in a sufficient amount to anesthetize the rodent. Some of the anesthetizing gas may then be conveyed from nose chamber 110 to waste gas reservoir 130 through gas transport channel 170. Some of the anesthetizing gas may flow from nose chamber 110 into incisor channel 114, and then into waste gas reservoir 130. This may be because of the open configuration between waste gas reservoir 130, incisor channel 114, and nose chamber 110. Ambient air may also flow into waste gas reservoir 130. The waste gas and ambient air may be actively retrieved from waste gas reservoir 130 with a negative flow conveyed through gas outtake channel 140 in communication with waste gas reservoir 130. This may be done in an amount sufficient to ensure that a human user of device 100 will be exposed to less than a maximum amount of the anesthetizing gas safe for human consumption.

It will be appreciated that any of a variety of anesthetizing gases may be used in accordance with the present disclosure. For example, the anesthetizing gas may comprise an anesthetizing agent such as isoflurane, enflurane, halothane, sevoflurane, nitrous oxide, desflurane, aliflurane, or any other inhalational anesthetic agents. The anesthetizing gas may also comprise an appropriate amount of oxygen delivered in conjunction with the anesthetizing agent. For example, isoflurane may be delivered in a ratio of approximately 2% isoflurane and approximately 98% oxygen. While other anesthetizing agents may use different ratios, the positive and negative flow rates may still be appropriate despite variations in the ratios of anesthetizing agent to oxygen.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. For example, various embodiments may perform all, some, or none of the steps described above. Various embodiments may also perform the functions described in various orders.

Although the present invention has been described above in connection with several embodiments; changes, substitutions, variations, alterations, transformations, and modifications may be suggested to one skilled in the art, and it is intended that the present invention encompass such changes, substitutions, variations, alterations, transformations, and modifications as fall within the spirit and scope of the appended claims.

What is claimed is:

1. A device for anesthetizing a rodent comprising:
a nose chamber configured to at least partially enclose an anesthetizing gas and to position and hold in contact with the anesthetizing gas a nose of the rodent;
a gas intake channel in communication with the nose chamber and configured to deliver a positive flow of the anesthetizing gas into the nose chamber, the positive flow sufficient to anesthetize the rodent;
a waste gas reservoir configured to collect the anesthetizing gas, the waste gas reservoir coupled with the nose chamber in an open configuration allowing the anesthetizing gas and ambient air to flow into the waste gas reservoir;
a gas transport channel configured to conduct the anesthetizing gas from the nose chamber to the waste gas reservoir; and
a gas outtake channel in communication with the waste gas reservoir and configured to convey a negative flow that actively retrieves the anesthetizing gas and ambient air from the waste gas reservoir.

2. The device of claim 1, wherein the positive flow of the anesthetizing gas into the nose chamber is regulated to flow at a controlled positive flow rate.

3. The device of claim 2, wherein the controlled positive flow rate is between about four hundred and fifty cubic centimeters per minute (450 cc/min) and about one thousand one hundred cubic centimeters per minute (1,100 cc/min).

4. The device of claim 2, wherein the controlled positive flow rate is about five hundred cubic centimeters per minute (500 cc/min).

5. The device of claim 1, wherein the negative flow actively retrieving the anesthetizing gas and the ambient air from the waste gas reservoir is regulated to flow at a controlled negative flow rate.

6. The device of claim 5, wherein the controlled negative flow rate is sufficient to ensure that a human operator of the device is exposed to less than a maximum amount of the anesthetizing gas acceptable for human consumption.

7. The device of claim 6, wherein the maximum amount of the anesthetizing gas acceptable for human consumption is two parts per million per hour (2 ppm/hr).

8. The device of claim 5, wherein the controlled negative flow rate is between about nine liters per minute (9 L/min) and twenty liters per minute (20 L/min).

9. The device of claim 5, wherein the controlled negative flow rate is about fifteen liters per minute (15 L/min).

10. The device of claim 1, wherein the gas intake channel further comprises a hose barb built onto an outer surface of the device to secure a hose configured to carry the positive flow of the anesthetizing gas.

11. The device of claim 1, wherein the gas outtake channel further comprises a hose barb built onto an outer surface of the device to secure a hose configured to carry the negative flow that actively retrieves the anesthetizing gas and ambient air.

12. The device of claim 1, wherein the waste gas reservoir comprises an open collection area encircled by a raised boundary and positioned directly beneath the nose chamber to collect heavier-than-air gases escaping from the nose chamber.

13. The device of claim 1, wherein the waste gas reservoir is configured to collect anesthetizing gas comprising at least one of:
directed anesthetizing gas conducted from the nose chamber to the waste gas reservoir through the gas transport channel,
escaped anesthetizing gas from the nose chamber, and
used anesthetizing gas exhaled from the rodent.

14. The device of claim 1, wherein the anesthetizing gas comprises isoflurane.

15. A method comprising:
positioning a nose of a rodent within a nose chamber of a device for anesthetizing the rodent, the nose chamber configured to at least partially enclose an anesthetizing gas and to hold the nose of the rodent in contact with the anesthetizing gas;
delivering a positive flow of the anesthetizing gas into the nose chamber through a gas intake channel sufficient to anesthetize the rodent;
supplying a waste gas reservoir to collect the anesthetizing gas, the waste gas reservoir in communication with the nose chamber through a gas transport channel and coupled with the nose chamber in an open configuration allowing the anesthetizing gas and ambient air to flow into the waste gas reservoir; and
actively retrieving the anesthetizing gas and ambient air from the waste gas reservoir with a negative flow conveyed through a gas outtake channel in communication with the waste gas reservoir.

16. The method of claim 15, wherein the positive flow of the anesthetizing gas is delivered at a controlled positive flow rate of between about four hundred and fifty hundred cubic centimeters per minute (450 cc/min) and about one thousand one hundred cubic centimeters per minute (1,100 cc/min).

17. The method of claim 15, wherein the negative flow actively retrieving the anesthetizing gas and ambient air from the waste gas reservoir is sufficient to ensure that a human operator of the device will be exposed to less than a maximum amount of the anesthetizing gas acceptable for human consumption.

18. The method of claim 15, wherein the negative flow actively retrieving the anesthetizing gas and ambient air from the waste gas reservoir is regulated to flow at a controlled negative flow rate of between about nine liters per minute (9 L/min) and about twenty liters per minute (20 L/min).

19. A system comprising:
a stereotactic surgical device; and
a device for anesthetizing a rodent coupled to the stereotactic surgical device, comprising:
a nose chamber configured to at least partially enclose an anesthetizing gas and to position and hold in contact with the anesthetizing gas a nose of the rodent;
a gas intake channel in communication with the nose chamber and configured to deliver a positive flow of the anesthetizing gas into the nose chamber, the positive flow sufficient to anesthetize the rodent;
a waste gas reservoir configured to collect the anesthetizing gas, the waste gas reservoir coupled with the nose chamber in an open configuration allowing the anesthetizing gas and ambient air to flow into the waste gas reservoir;
a gas transport channel configured to conduct the anesthetizing gas from the nose chamber to the waste gas reservoir; and
a gas outtake channel in communication with the waste gas reservoir and configured to convey a negative flow that actively retrieves the anesthetizing gas and ambient air from the waste gas reservoir.

20. The system of claim 19,
wherein the stereotactic surgical device comprises an incisor bar and the device comprises an incisor channel; and
wherein the device is coupled to the stereotactic surgical such that the incisor bar passes through the incisor channel.
